(19) United States
(12) Patent Application Publication
(10) Pub. No.: US 2005/0004580 A1
(43) Pub. Date: Jan. 6, 2005

(54) System for pointing a lesion in an X-rayed object

(76) Inventors: Tommi Jokiniemi, Hyvinkaa (FI); Timo Ihamaki, Vantaa (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 10/611,836

(22) Filed: Jul. 1, 2003

(51) Int. Cl. A61B 19/00

(52) U.S. Cl. 606/130

(57) ABSTRACT

A method for pointing a suspected lesion in an X-rayed body portion of a human or animalian body is disclosed, whereupon the body portion is clamped in a fixed position on a platform having a radiographic imaging detector, and radiated with X-rays coming successively from at least two different directions to form at least two planar images and respective image data. From said at least two image data and from said at least two directions, an inside location of the lesion is calculated in a predetermined three-dimensional coordinate system having two coordinate values in a plane substantially parallel to said platform, and further the configuration of the tissue surface is estimated. Then an entering point for an invasive instrument is selected and a moving direction for said invasive instrument is determined, together with calculating the distance between said entering point on said estimated surface and said calculated inside location in said moving direction. Finally the moving direction and the distance is used for guiding said invasive instrument.

### FIELD OF THE INVENTION

The present invention relates to a method for pointing a suspected lesion in an X-rayed body portion of a human or animalian body in order to facilitate taking of a biopsy sample reliably and precisely from the suspected lesion, and enabling a more precise marking of the lesion.

### BACKGROUND OF THE INVENTION

Panoramic and tomographic imaging systems are widely used for attaining images from target areas of human and animalian bodies, and nowadays these systems are also used for taking three-dimensional X-ray photographs from target volumes of human and animalian bodies, whereupon solid-state detectors like radiation sensitive semiconductor sensor, e.g. CCD-sensors or other kind of sensor systems producing digital image data are typically utilized. It is generally known that, for instance, a precise enough insertion of a biopsy needle for taking a sample of a lesion in the human or animalian tissue suspected to be a tumor in order to determine whether it is malignant or benign, or a precise enough insertion of indicator wires into human or animalian tissue for marking the detected tumor prior to surgical operation is a demanding operation. When the X-rays are utilized for detection of the lesion, the body part like the breast of the female patient, inside which the suspected lesion is located, is compressed against a surface below which an X-ray film or a detector is positioned. A compression plate is placed above the breast, clamping it against the surface, but leaving a tissue surface area exposed because of a larger opening in the plate. In the widely used process the opening has indicia along its sides, and after determining the two orthogonal coordinate values of the location of the lesion from the X-ray image, which coordinate values are in plane parallel to the above-mentioned surface, a mark is placed on the respective location on the exposed skin by utilizing the indicia. Another X-ray image is taken to show the depth of the lesion and to assure that the biopsy is taken from the proper position. This is a quite unreliable and time-consuming procedure, which may also require several attempts to hit the intended lesion.

Patent publication U.S. Pat. No. 4,727,565 discloses a slightly alternative method for localizing the three dimensional position of a spot in an object in conjunction with the X-ray exposure of said object. According to the method the object is clamped in a predetermined position, whereafter a first print of at least said spot in said object by exposure of the object to a source of X-rays in a first direction from a first position on one side of a center line at right angles to the image plane of the first print is obtained, providing a first index on said first print. Then, with the object remaining clamped in the same predetermined position, a second print of said spot by exposure of the object to said source of X-rays in a second direction from a second position on the other side of said center line is obtained, providing a second index on the second print, establishing the two-dimensional position of the spot on the two image prints in relation to the index on the prints. Finally the coordinates of the spot in relation to the indices is processed for determination of the three dimensional position of said spot so as to enable control of a guidance instrument to the spot located in the object. Patent publication U.S. Pat. No. 4,930,143 discloses a substantially analogous method for stereographic location in a breast of a lesion suspected of being cancerous using a mammographic unit comprising an X-ray tube mounted on the stand so as to emit an X-ray beam in a defined field; a holder, laterally slidable, with respect to said beam, between a first imaging position within said field and a second imaging position within said field, for receiving a breast and restraining the breast in a fixed shape, and means for holding a film, located within the X-ray field and held stationary relative to the X-ray tube, in a position such that a first picture of said breast taken at the first imaging position and a second picture of the breast taken at the second imaging position will be located side by side on the film. According to the disclosed method a breast is placed in the holder, restraining the breast in a fixed shape; the breast is exposed to X-rays while the holder is in said first imaging position so that a first picture is made on the film. Then the holder is slid to said second imaging position while restraining the breast in the fixed shape thereof; the breast exposed to X-rays while the holder is in its second imaging position so that a second picture is made on the film adjacent the first picture. Finally said first and second pictures are used to calculate the perceived parallactic displacement of a lesion imaged on the film; and on the basis of said displacement the position of the lesion within the breast is determined. Accordingly, these publications concern determining the exact position of a lesion, according to U.S. Pat. No. 4,727,565 the object is kept stationary and the X-ray source is moved, and according to U.S. Pat. No. 4,930,143 the X-ray source is kept stationary and the object is moved, to attain a pair of stereo pictures, but neither of them discuss the problem of inserting the biopsy needle or marking wire precisely into the detected lesion. Patent publication U.S. Pat. No. 5,107,843 discloses an apparatus for locating a needle for thin needle biopsy. For the purpose a mammography apparatus is used, which apparatus includes a rotating picture head to obtain two pictures of a biopsy target, a detachable needle guide that can be attached to a fixed attachment means in said mammography apparatus and a separate measurement table. The separate measurement table has a fixed attachment means for said needle guide, and measuring means on said measurement table for measuring and calculating the orthogonal x, y and z coordinates of said biopsy target from said two pictures, wherein said fixed attachment means on said measurement table and said fixed attachment means on said mammography apparatus are located in the same position with respect to coordinates calculated from said two pictures of said biopsy target. So, according to this publication the coordinates of the lesion are the output and the operator like physician must rely on these three values and the needle guide, which are extremely illogical and non-intuitive for the operator, easily causing errors and additional attempts to hit the lesion.

Patent publication U.S. Pat. No. 5,316,014 discloses apparatus for use in X-ray examination and diagnostic procedures comprising an X-ray machine having an X-ray radiation head mounted in spaced relation to a patient and specimen supporting platform for supporting a specimen, a clamping means mounted in spaced parallel relation to said platform to clamp the specimen against said platform and against movement relative to the X-ray machine. The clamping means have an opening exposing a portion of the specimen and indicia associated with the clamping means for locating a lesion on an X-ray picture by means of coordinates showing in the X-ray pictures taken by the X-ray radiation head. A laser head is detachably supported on said X-ray machine between said X-ray radiation head and the specimen supporting platform, whereupon first and second laser sources are mounted in said laser head radiating focused beams in first and second planes intersecting along an intersecting line and providing cross lines intersecting on said portion of the specimen and lying within said opening. The apparatus further comprises means for moving the laser head to shift the laser head and the cross lines to a position in accordance with the coordinates location of the lesion as shown by the X-ray picture. The other end of a biopsy needle, to be inserted into the specimen, being away from the specimen receives the cross lines, which appear as a dot for locating the axis of said needle with respect to said specimen during insertion of the needle for removal of a core sample.

A problem associated with this biopsy is the difficulty of inserting and guiding the needle at the correct angle so that the needle tip is not displaced to a side of the tumor when the needle is inserted to the proper depth. If the needle tip is inserted only along a true vertical plane, there is a chance, because of the parallax between the true direction of the X-ray through the lesion and said vertical plane, that the needle tip may be displaced at an angle from the lesion missing the lesion. To avoid this risk the patent publication U.S. Pat. No 5,320,111 suggests, as an supplement to the system of patent publication U.S. Pat. No. 5,316,014, a generation of a laser beam in a continuous cross hair pattern emanating from a location along the line of the X-ray radiation, positioning the continuous laser beam in accordance with the coordinate locations of said lesions as shown by the X-ray picture, and adjusting the inclination of the laser beam for different coordinate locations of the lesion and directing the needle along these different inclination at each the respective locations. The tip of a biopsy needle is applied to the intersection on the specimen of the lines formed by the laser beam. More specifically, the laser source is moved to eliminate parallax and to guide the needle along the angle and to the position of the tumor to assure that the needle be inserted at the same angle as the X-ray beam from the X-ray point source.

The main object of the invention is to attain a method, through which a suspected lesion inside a body portion of a patient can be pointed or indicated so that a swift and accurate reaching of the suspected lesion is allowed for e.g. the physician. Another object of the invention is to attain a method, which enable insertion of the biopsy or puncture needle or insertion of the marking wires in direction(s) other than the direction of the X-rays, if wanted. The third object of the invention is to attain a method, which enable automation of the pointing or indicating procedure, and through which use of expensive separate parts with indicia, like such compression plates provided with coordinate markings, can be avoided. Further object of the invention is to attain a method, which is as comfortable as possible to the patient, whereupon unnecessary delays and repeating should be avoidable.

### SUMMARY OF THE INVENTION

According to the first aspect of the invention it is provided a method for pointing a suspected lesion in an X-rayed body portion of a human or animalian body, the method comprising the steps: clamping said body portion in a fixed position on a platform provided with a radiographic imaging detector, said body portion having a substantial non-compressed tissue surface area apart from said platform towards an X-ray source, and the suspected lesion having an inside location within said body portion; radiating said body portion with X-rays coming successively from at least two different directions to form at least two planar images and respective image data of said body portion; calculating, from said at least two image data and from said at least two directions, said inside location in a predetermined three-dimensional coordinate system having two coordinate values in a plane substantially parallel to said platform; estimating a configuration of said tissue surface from said image data; selecting an entering point for an invasive instrument within said surface area; determining a moving direction for said invasive instrument; calculating a distance between said entering point on said estimated tissue surface and said calculated inside location in said moving direction; and displaying or outputting said two coordinate values, said moving direction and said distance for guiding said invasive instrument.

According to the second aspect of the invention it is provided a method for pointing a suspected lesion in an X-rayed body portion of a human or animalian body, the method comprising the steps: clamping said body portion in a fixed position on a platform provided with a radiographic imaging detector, said body portion having a substantial non-compressed tissue surface area apart from said platform towards an X-ray source, and the suspected lesion having an inside location within said body portion; attaching at least one marker on said tissue surface area to have an outside location; radiating said body portion with X-rays coming successively from at least two different directions to form at least two planar images and respective image data of said body portion; deriving inside location data and outside location data from said at least two image data and from said at least two directions; calculating said inside location in a predetermined three-dimensional coordinate system from said inside location data with two coordinate values in a plane substantially parallel to said platform; estimating a configuration of said tissue surface from said outside location data; selecting an entering point for an invasive instrument within said surface area; determining a moving direction for said invasive instrument; calculating a distance between said estimated tissue surface and said calculated inside location in said moving direction; and displaying or outputting said two coordinates, said moving direction and said distance for guiding said invasive instrument.

According to the third aspect of the invention it is provided a method for pointing a suspected lesion in an X-rayed body portion of a human or animalian body, the method comprising the steps: clamping said body portion in a fixed position on a platform provided with a radiographic imaging detector, said body portion having a substantial non-compressed tissue surface area apart from said platform towards an X-ray source, and the suspected lesion having an inside location within said body portion; attaching at least one marker on said tissue surface to have an outside location; radiating said body portion with X-rays coming successively from at least two different directions to form at least two individual images and respective image data of said body portion; deriving inside location data and outside location data from said at least two image data and from said at least two directions; calculating a direction and a respective distance between said marker and said calculated inside location for entering an invasive instrument; and displaying or outputting said direction and said distance for guiding said invasive instrument.

According to the fourth aspect of the invention it is provided a method for pointing a suspected lesion in an X-rayed body portion of a human or animalian body, the method comprising the steps: clamping said body portion in a fixed position on a platform provided with a radiographic imaging detector, said body portion having a substantial non-compressed tissue surface area apart from said platform towards an X-ray source, and the suspected lesion having an inside location within said body portion; radiating said body portion with X-rays coming from at least a first direction to form at least a first individual image and respective image data of said body portion; deriving inside location data from said at least first images and from said at least first direction; calculating said inside location in a predetermined two-dimensional coordinate system from said inside location data with coordinate values in a plane substantially parallel to said platform; displaying or outputting said two coordinates for guiding said invasive instrument; determining a moving direction for an invasive instrument having a tip; radiating said body portion, after inserting an invasive instrument into said body portion or in contact or approaching a contact with said tissue surface, with X-rays coming from at least a second direction to form at least a second individual image of said body portion; measuring a spacing between said tip and said suspected lesion from said second image; calculating, from said spacing and from said second direction, a distance between said tip and said suspected lesion in said moving direction; and displaying or outputting said distance and said moving direction for guiding said invasive instrument.

According to the fifth aspect of the invention it is provided a method for pointing a suspected lesion in an X-rayed body portion of a human or animalian body, the method comprising the steps: clamping said body portion in a fixed position on a platform provided with a radiographic imaging detector, said body portion having a substantial tissue surface area apart from said platform and compressed by a compression plate substantially transparent to X-rays and having a plurality of perforations towards an X-ray source, and the suspected lesion having an inside location within said body portion; radiating said body portion with X-rays coming successively from at least two different directions to form at least two individual images and respective image data of said body portion and of said perforated plate; deriving inside location data and perforated plate location data from said at least two image data and from said at least two directions; selecting at least one perforation in said plate and determining a moving direction for an invasive instrument through said at least one perforation; calculating a distance between said at least one perforation of said plate and said calculated inside location in said moving direction of the invasive instrument; and displaying or outputting said at least one perforation, said direction and said distance for guiding said invasive instrument.

According to the sixth aspect of the invention it is provided a method for pointing a suspected lesion in an X-rayed body portion of a human or animalian body, the method comprising the steps: clamping said body portion in a fixed position on a platform provided with a radiographic imaging detector, said body portion having a substantial tissue surface area apart from said platform and compressed by a compression plate substantially transparent to X-rays and having a plurality of perforations towards an X-ray source, and the suspected lesion having an inside location within said body portion; radiating said body portion with X-rays coming from at least a first direction to form at least an individual image and respective image data of said body portion and of said perforated plate; selecting a perforation in said plate and determining a moving direction for an invasive instrument having a tip through said at least one perforation; radiating said body portion, after inserting said invasive instrument, with X-rays coming from at least a second direction to form at least another individual image and respective image data of said body portion and of said perforated plate and said invasive instrument; calculating a distance between said tip of the invasive instrument and said calculated inside location in said moving direction of the invasive instrument; and displaying or outputting said distance for further guiding said invasive instrument.

### BRIEF DESCRIPTION OF THE DRAWINGS

The foregoing summary, and the following detailed description of the preferred embodiments of the present invention, will be better understood when read in conjunction with the accompanying drawings, in which:

FIG. 1A illustrates generally an X-ray apparatus with a tilting X-ray head, which allows radiating the body portion of the patient, a breast—in a position which is actually not used in practice because of the visibility—in the case shown, from different directions, and which can be utilized for the method according to the present invention, in an axonometric view.

FIG. 1B illustrates the X-ray apparatus of FIG. 1A with a functioning depth indicating light beam device integrated in the compression plate moving unit, and a functioning biopsy needle directing light beam device mounted or turned in its operating position or integrated in the tilting X-ray head, in the same view as in FIG. 1A.

FIGS. 2A and 2B illustrate the detection of a lesion and the guidance principle for insertion of e.g. a biopsy needle, when the first embodiment of the method according to the invention is utilized. FIG. 2A is a cross-section of a body portion of the patient, like a breast, and the related means, in the vertical plane I-I of FIG. 1A; and FIG. 2B is a plan view to the body portion area of FIG. 2A in the direction II of FIG. 2A.

FIG. 3 illustrates the detection of a lesion and the guidance principle for insertion of e.g. a biopsy needle, when the second embodiment of the method according to the invention is utilized. FIG. 3 is a cross-section of a body portion of the patient, like a breast, and the related means, in the respective vertical plane as in FIG. 2A.

FIG. 4 illustrates the detection of a lesion and the guidance principle for insertion of e.g. a biopsy needle, when the third embodiment of the method according to the invention is utilized. FIG. 4 is a cross-section of a body portion of the patient, like a breast, and the related means, in the respective vertical plane as in FIGS. 2A and 3.

FIG. 5 illustrates the detection of a lesion and the guidance principle for insertion of e.g. a biopsy needle, when the fourth embodiment of the method according to the invention is utilized. FIG. 5 is a cross-section of a body portion of the patient, like a breast, and the related means, in the respective vertical plane as in FIGS. 2A, 3 and 4.

FIGS. 6A to 6C illustrate the detection of a lesion and the guidance principle for insertion of e.g. a biopsy needle, when the fifth embodiment of the method according to the invention is utilized. FIG. 6A is a cross-section of a body portion of the patient, like a breast, and the related means, in the respective vertical plane as in FIGS. 2A, 3, 5 and 5. FIG. 6B is an enlarged view of the area III of FIG. 6A visualizing the perforated plate in the same cross section, and FIG. 6C is a plan view to the plate area of FIG. 6B in the direction IV of FIG. 6B.

FIG. 7 exemplifies graphically radiation intensity distributions over one linear section of the detector received when radiated from two different directions, from which intensity distribution the positions of the lesions can be calculated and the configuration of the outer surface of the body portion can be estimated. Distribution shown is from section A-B of FIG. 2B.

FIG. 8 illustrates one alternative method according to the invention for giving guidance to the operator in manual biopsy needle insertion, whereupon an extended laser beam cross is used for e.g. positioning in horizontal directions and for tilt angle control, and a flabellate laser beam is used for controlling the insertion depth of the biopsy needle; in an axonometric view.

FIG. 9 illustrates two other alternative methods according to the invention: For giving guidance to the operator in semi-automatic biopsy needle insertion provided with needle position detectors and a display/control unit; And for automatic biopsy needle insertion provided with needle insertion servos and a control unit; in an axonometric view.

FIGS. 10 and 11 represent a biopsy needle with length indicia, and several biopsy needles having various lengths respectively, which can be utilized for controlling the depth of insertion.

FIG. 12 is a flow chart disclosing the main steps of the first and second embodiments of the method according to the invention, corresponding to the arrangements of FIGS. 2A to 3.

FIG. 13 is a flow chart disclosing the main steps of the third and fourth embodiments of the method according to the invention, corresponding to the arrangements of FIGS. 4 and 5.

FIG. 14 is a flow chart disclosing the main steps of the fifth embodiment of the method according to the invention, corresponding to the arrangements of FIGS. 6A to 6C.

### DETAILLED DESCRIPTION OF THE PREFERRED EMBODIMENTS

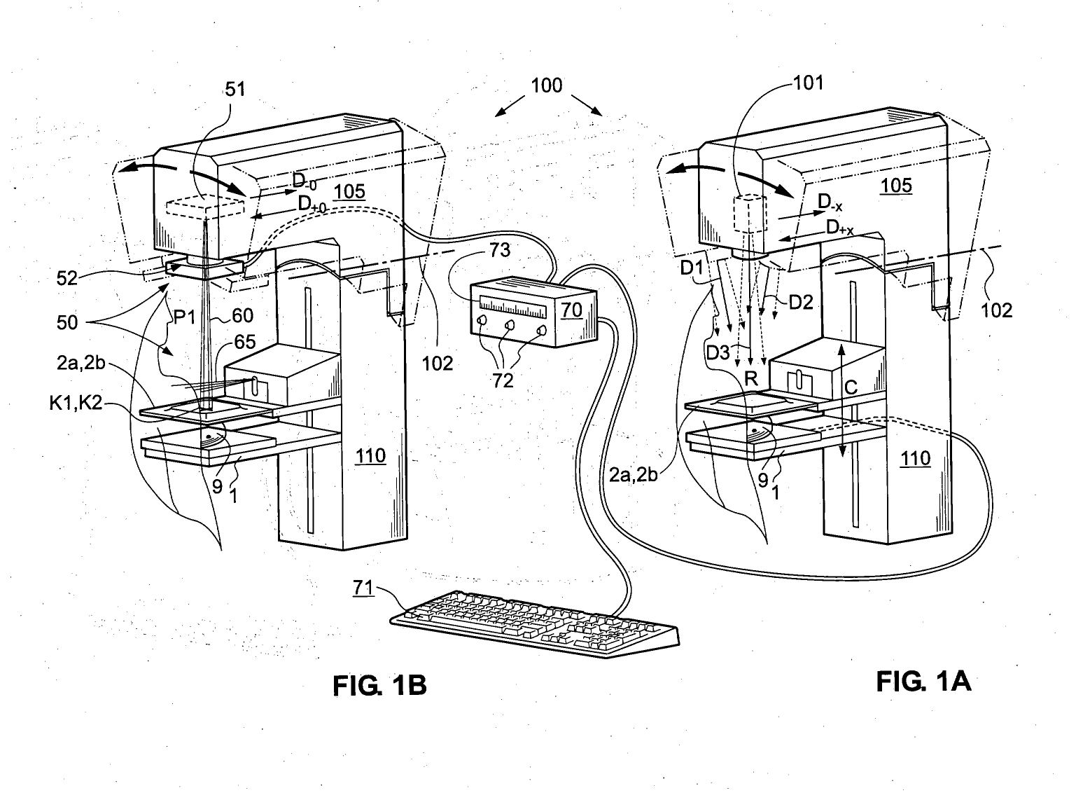

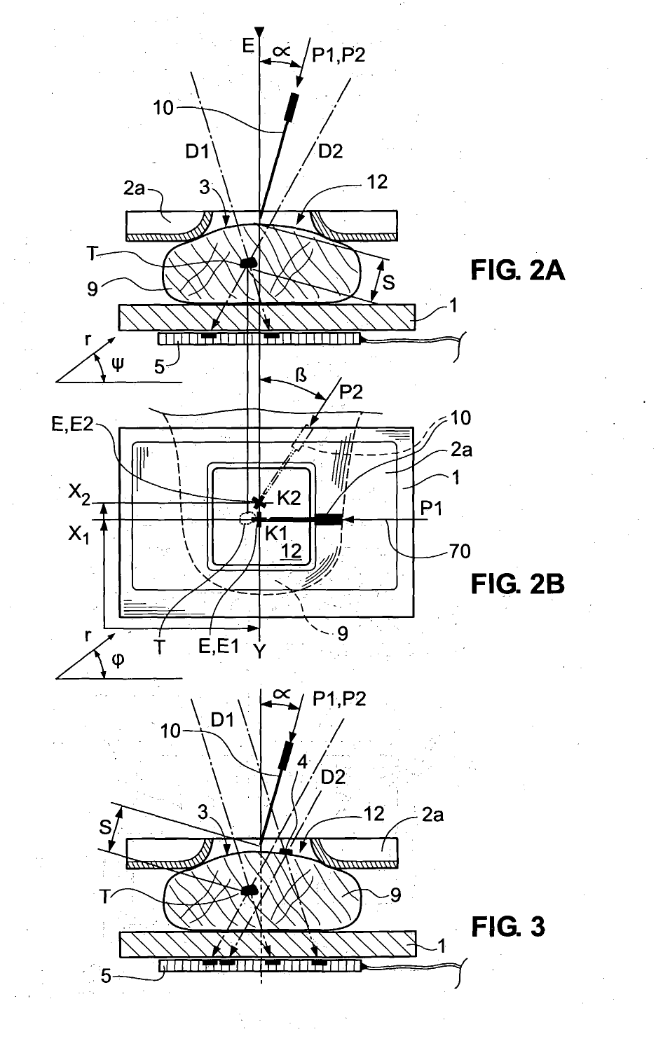

FIGS. 1A and 1B show the general features of an X-ray apparatus 100. The apparatus 100 has a frame 110 and means for clamping the body portion 9 of a patient a fixed position. The means for this clamping comprise a platform 1 and a compression plate 2a or 2b, between which the body portion, i.e. the target, to be X-rayed is positioned and clamped. The platform is provided underneath with a radiographic imaging detector 5, typically a CCD-detector or a CMOS-detector having a plurality of image pixels forming an X-ray sensitive area, as visible in FIGS. 2A to 6A. The X-ray source 101 is within a head 105 of the apparatus, and emits the X-ray beam towards the compression plate 2a, 2b, through the body portion 9, through the platform 1 or some part thereof, and falls into the imaging detector 5. The image detector 5 transforms the received radiation R into electrical signal, which is transferred as image data to a computer for further processing. In this application, the definition “image” means any data carrying information about the target body portion respective to an image. Image data can be also called virtual image. There is no need to show the image or images as a concrete or physical print or on a display, even if the images can be printed or displayed if wanted, but the display or output of that information specific to the present invention is all that is necessitated. This kind of image processing is familiar to any person skilled in the art, and is not described in detail. The head 105 with the X-ray source 101 is tiltable around an axis 102 in respect to the frame 110 and the combination of the platform and the compression plate, so as to enable directing the X-ray beam from different directions D1, D2, D3 to the body portion 9. Radiating a target, like a body portion, at least from two directions or from several directions more than two, whereupon each radiation direction D1, D2, D3 produces a separate planar image, i.e. a planar virtual image, is generally known, and accordingly, neither the apparatus nor the data processing is not described in detail. The two or more planar images taken from a single immobilized target 9 serve as the starting point for configuring through calculations a three-dimensional image data, i.e. a virtual three-dimensional image, which configuration or calculation can be performed using a computer and a proper program. The data processing for attaining a three-dimensional image data is generally known, this kind of data processing as such is not the object of the invention, and accordingly this further processing is neither described in detail. It shall be, however, be mentioned that a three-dimensional image data includes information about the inner structure of the body portion together with the data about the positions of the structural details. The three-dimensional image, which can be also called as stereo image, based on the data can be shown e.g. on a display, not shown in the figures, so that a physician or a nurse or other specialist, generally an operating person, can evaluate it and make necessary decisions and operations. Displaying and evaluation of the three-dimensional images or stereo images as such are not the subject of the present invention, though the same image data is utilized for it, as described later. Anyway, the suspected lesion T, if found, has an inside location within the body portion 9, or the suspected lesions, if found several, have inside locations within said body portion, and then a sample shall be taken from each of them using biopsy or puncture, or those to be removed surgically shall be marked by wires or dye.

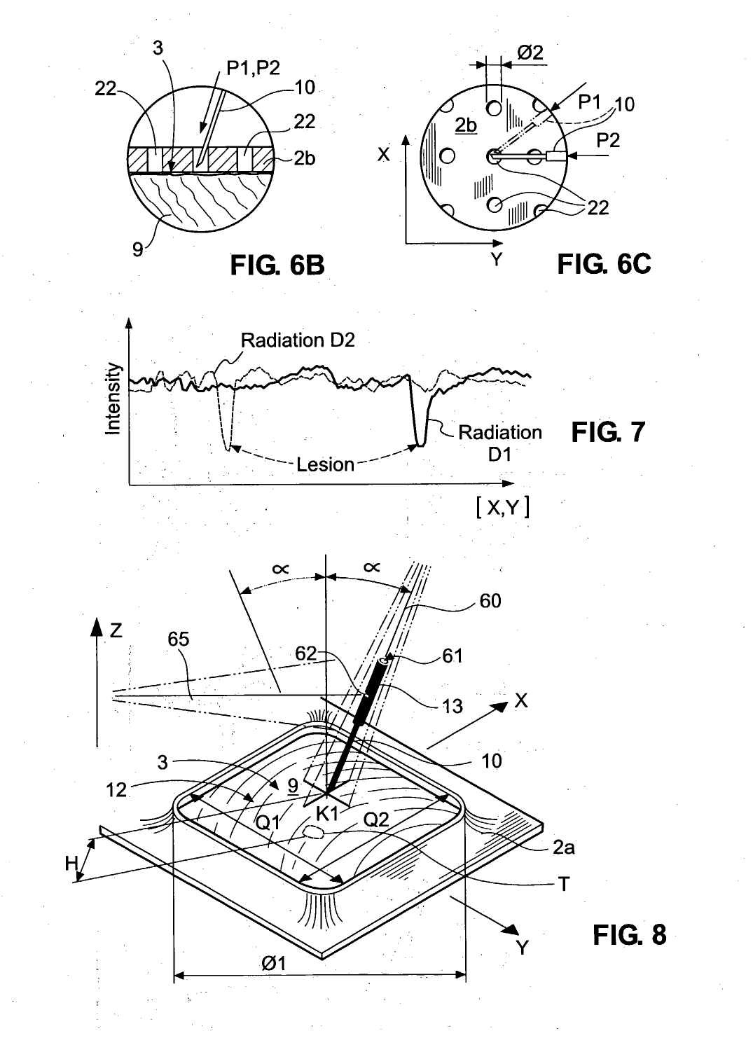

The body portion 9 of the patient is especially a breast of a woman, whereupon it is mammography in question, but in principle the body portion can be any projecting body part, like arm or leg etc., which can be at least partly compressed between the compression plate 2a, 2b and the platform 1 to maintain the body portion unmovable for the period of imaging and taking a biopsy or a puncture or inserting marking wires for surgical operation. Hereinafter, the definition biopsy is used to mean taking both liquid and tissue samples. It shall be understood that the very function of the compression plate is to immobilize the body portion and that for this purpose the “plate” can also have a form not actually a plate, but e.g. a trough or any configuration practical for the purpose. The definition “compression plate” is accordingly used here for simplicity only, because in mammography an at least partly plate-like member is typically used, and not for limitation. Any configuration of the plate is included in the scope. Often, but not necessarily, this compression plate 2a has a larger opening 12, as shown in FIGS. 2A to 5 and 8 to 9, to enable accessibility to taking a biopsy or inserting marking wires into the lesion. This large opening 12 causes that the body portion, in this case the breast, has a substantial non-compressed tissue surface area apart from said platform towards the X-ray source 101. The non-compressed area bulges upwards, i.e. away from the platform 1, because of the elastic properties of the tissue, whereupon the exact position of tissue surface varies from case to case. The opening 12 shall be considered large, when this bulging may cause such variations in the position of the tissue surface that makes the precise enough hitting of the lesion with the invasive instrument 10, like biopsy needle or marking wire, too difficult or impossible. It is believed that the opening 12 is large, when its diameter Ø1 is larger than 1 cm or it has dimensions Q1, Q2 larger than 1 cm×1 cm. In the embodiment of FIGS. 6A to 6C the compression plate 2b has a plurality of smaller perforations 22, having a diameter Ø2 or other respective dimensions substantially smaller than 1 cm, but larger than the diameter Ø3 of the invasive instrument 10 allowing insertion of the invasive instrument through a perforation. In this case there is no non-compressed tissue surface area, because the extremely small bulges at the small areas of perforations can be neglected. Compression is performed by moving the compression plate 2a or 2b in a direction C, e.g. parallel to coordinate direction z, perpendicular to the platform 1. There are several tasks in order to assure that marking of a lesion by wires is performed accurately enough, or taking a sample from a lesion, i.e. biopsy, is obtained accurately enough. The position of the lesion T shall be determined in three coordinate directions x, y, z, in case of orthogonal coordinate system, or in three coordinate directions r, φ, ψ, in case of polar coordinate system. The direction and the distance from the selected entering point of the invasive instrument 10 into the lesion shall be calculated. The selected entering point E on the outer surface 3 of the body portion 9 shall be either indicated prior to the contact between the invasive instrument and the outer surface, or traced after the entering point E is selected by the first contact between the invasive instrument and the outer surface radiating. Then the moving direction P1, P2, indicated by at least a tilt angle a and if necessary also by a turn angle β, and the distance S of the invasive instrument shall be controlled; either manually using displayed values or mechanically by automatic drives using output values. The tilt angle α, and the possible turn angle β, of the guiding light beam 60 are not the same as the parallax of the X-rays, but directions totally independent from the X-ray parallax.

In all embodiments of the invention the body portion 9 is radiated with X-rays R coming successively from at least two different directions D1 and D2 and/or D3 to form at least two planar images of the body portion, i.e. two stereoscopic images, whereafter a three-dimensional image data is derived from said at least two data sets that correspond the planar images formed on the imaging detector and from said at least two directions. This 3D-image data carries information about the internal structure of the body portion, and accordingly the inside location(s) of the lesion(s), i.e. inside location data=coordinates of the lesion inside the body portion, can be calculated from that image data in a predetermined three-dimensional coordinate system, which has two coordinate values r, φ, or preferably x, y in a plane substantially parallel to the platform 1, and coordinate value z or ψ in direction perpendicular to the platform. Also the outside location=coordinates of the different points of the tissue surface 3 of the body portion can be calculated from that image data in the predetermined three-dimensional coordinate system. FIG. 7 exemplifies two intensity distributions as received by the detector 5 across one line and forwarded as electrical signals carrying detected intensity data, i.e. image data, to a computer unit 70. It shall be understood that the detector receives and forwards intensity data also from a plurality of neighboring lines, whereupon the area of the detector is utilized. The first intensity distribution corresponds data received when radiated from direction D1 and the second intensity distribution corresponds data received when radiated from direction D2. As can be seen the intensity drops caused by one lesion T are in positions spaced apart from each other, and is known the position of the lesion in the vertical direction and in the direction of this exemplary line can be calculated. In actual occasion when neighboring lines are also available the position of the lesion can be calculated also in the direction perpendicular to surface of the figure, as can be readily understood. The tissue surface can be calculated from varying intensity outside and between said intensity drops, though the calculations are more complicated.

In the arrangement of FIGS. 2A and 2B the configuration of the tissue surface 3 is estimated without any markers or invasive instruments, but its form and position is calculated solely from the image data received by the image detector and the radiation directions. This is possible because the atmosphere at any point above the body portion deviates from any point inside the body portion, causing traces in the two-dimensional image data and accordingly in the three-dimensional image data, too. On the basis of the inside location data an entering point E for the invasive instrument 10 within the accessible area, that is in the area of the opening 12, of the surface 3 is selected. This selection can be done in three ways: {A} The optimum entering point can be calculated from the image data and indicated—as described later in detail—for the operating person; Or {B} the operating person can select the entering point on the basis of displayed image(s) or other data using personal experience and evaluation, feed the intended data into the computer, whereafter the selected point is indicated for the operating person; Or {C} the operating person can select the entering point on the basis of displayed image(s) or other data using personal experience and evaluation, position the invasive instrument in the selected position on the tissue surface 3, whereafter the selected entering point is traced—as described later in detail—e.g. by optical means. When the entering point E is finalized, the intended and optimum moving direction P1, P2 of said invasive instrument is determined using the three coordinates of the entering point E, which are calculated from the estimated configuration of the tissue surface 3 including the entering point, and the three coordinates of the lesion T calculated earlier. Then the distance S and the moving direction P1, P2 between two points, i.e. from the entering point E on said estimated surface and to the calculated inside location of the lesion T, can be readily calculated. In the alternative {C} the manually selected and then traced entering point E is no more calculated, but is kept in the prevailing position, and the moving direction P1 or P2 and the distance S are output, i.e. forwarded to a instrument guide device 30, like a position motor means, or guiding said invasive instrument. In the alternatives {A} and {B} the automatically selected, or the manually selected and to the computer fed entering point E is displayed or output, i.e. forwarded to an instrument guide device 30, using the two coordinate values thereof, and the moving direction P1 or P2 and said distance S are also displayed or output, i.e. forwarded to an instrument guide device 30, for guiding said invasive instrument.

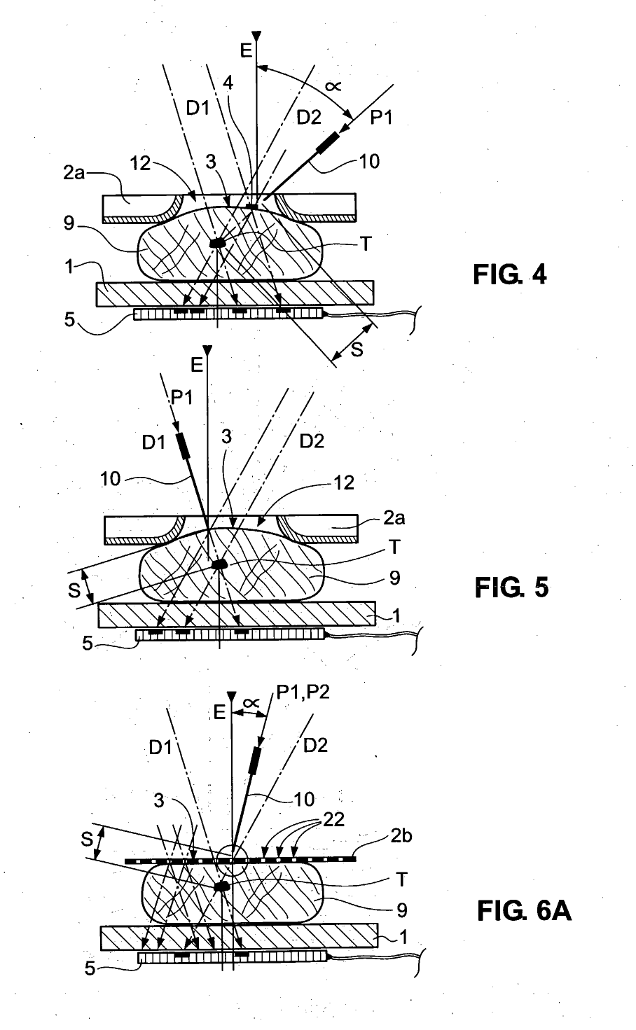

In the arrangement of FIGS. 3 and 4 at least one marker 4 is attached, according to one alternative, on the tissue surface 3 to have an outside location prior to radiating the body portion with X-rays. The marker 4 or markers has/have high radiation absorption to X-rays, and so leave an extremely clear trace in the two-dimensional image data, whereafter the exact position of the outer surface 3 at this/these spot(s), i.e. outside location data, can be calculated from the images and the configuration of surface 3, also in points other than the markers, can be mathematically estimated using a proper algorithm from the outside location data. In the arrangement of FIG. 3 the further operations are exactly the same as described above in the context of FIGS. 2A and 2B. In the arrangement of FIG. 4 the further operations are otherwise the same as above, but the position of the marker 4 is used as the entering point E, whereupon the invasive instrument 10 is inserted just on the side of the marker. The direction P1, P2 and the respective distance S between the marker and the calculated inside location of the lesion T are the values, which shall be calculated and displayed or output, i.e. forwarded to a instrument guide device 30, like a position motor means, for guiding said invasive instrument. In this latter case the marker or entering point is may not be optimally positioned, because it is attached prior to imaging. On the other hand its position data is very precise and there is no need to display its position to the operating person, because it is readily visible without any further indicating means, though the display of the two coordinate values cannot be avoided because of the display of the direction(s) inevitably includes also these indications. For the arrangement of FIG. 4, another alternative can be utilized, in which the marker is attached after radiating the body portion with X-rays for e.g. the first time. In this case the radiation of the body portion 9 with X-rays coming from at least a first direction D1 produces the first planar image, from which the inside location data can be derived concerning the position of the lesion in the coordinate values parallel to the platform 1. When these coordinate values are displayed to the operating person the marker 4 can be positioned properly. When radiating the body portion with X-rays coming from at least a second direction D2, whereupon at least a second individual planar image of said body portion is formed. Then measuring the distance S and the moving direction P1, P2 between the marker, and simultaneously between the tip of the invasive instrument and said suspected lesion T from said second image can be calculated. Finally, the distance S and the moving direction P1, P2 are displayed or output, i.e. forwarded to an instrument guide device 30, for guiding said invasive instrument.

The arrangement of FIG. 5 is in principle very close to the arrangement of FIGS. 3 and 4, the difference being that the invasive instrument 10 itself acts as the marker. According to one alternative the invasive instrument is inserted in contact with said tissue surface 3, and supported with an X-ray transparent means to stay in its position, prior to radiating the body portion with X-rays, whereupon the tip 11 of the invasive instrument on the tissue surface 3 has an outside location, as above. Further steps are the same as described in the context of FIG. 4. According to another alternative the invasive instrument is inserted in contact with said tissue surface 3 or inserted into said body portion, and supported with an X-ray transparent means to stay in its position, after radiating the body portion with X-rays for e.g. the first time, whereupon the tip 11 of the invasive instrument on the tissue surface 3 has an outside location. In this case the radiation of the body portion 9 with X-rays coming from at least a first direction D1 produces the first planar image, from which inside location data can be derived concerning the position of the lesion in the coordinate values parallel to the platform, whereupon the inside location can be calculated in a predetermined two-dimensional coordinate system with coordinate values in a plane substantially parallel to said platform 1 and the moving direction P1, P2 for an invasive instrument 10 can be determined. When these coordinate values are displayed to the operating person the invasive instrument can be positioned properly, and preferably so as to point to the lesion T and the tip 11 of the invasive instrument closer the lesion than the tissue surface. When radiating the body portion with X-rays coming from at least a second direction D2, whereupon at least a second individual planar image of said body portion is formed. Then measuring the spacing between said tip 11 and said suspected lesion T from said second image is straightforward operation, and further the distance S between said tip and said suspected lesion in said moving direction P1, P2 can be calculated from said spacing and from said second direction. Finally, the distance S is the only value that shall be displayed or output, i.e. forwarded to an instrument guide device 30, for guiding said invasive instrument.

In the arrangement of FIGS. 6A to 6C the body portion 9 has a substantial tissue surface area apart from said platform and compressed by a compression plate 2b substantially transparent to X-rays. Here the compression plate 2b has an area that is in contact with tissue surface 3 determining typically a planar configuration to the tissue surface and the vertical position thereof. This compression plate is provided with a plurality of perforations 22 open towards an X-ray source, which perforations are small as described above causing no such bulging of tissue that should be taken account. The body portion is radiated with X-rays coming successively from at least two different directions D1, D2 to form at least two individual images of the body portion and of the perforated plate, followed by deriving the inside location data and perforated plate location data from said at least two images and from said at least two directions. The selection of the entering point of the invasive instrument 10 is performed in an analogous way as compared to the embodiments of FIGS. 2A to 3, with that exception that the entering point is finally one perforation of the compression plate. The selection of the single perforation 22 can be done automatically by a program in a computer, or manually and fed into the computer or traced by the guiding light beam and then fed to the computer. On the basis of the determined single perforation and the known position of the lesion the moving direction of the invasive instrument through said at least one perforation can be determined, and further the distance between said at least one perforation of the plate and the calculated inside location in said moving direction of the invasive instrument can be calculated. This calculated direction P1, P2 and the distance S are finally displayed, or output, i.e. forwarded to an instrument guide device 30, for guiding said invasive instrument.

FIG. 1B show the general principle of the display and optical guide arrangement according to the invention, which are used for informing the operating person about the two coordinate values of the entering point E parallel to the platform, or for tracing the two coordinate values of the entering point E parallel to the platform selected manually by the operating person, and informing the operating person about the moving direction P1, P2 described by the tilt angle α and possibly by the turn angle β together with the moving distance S of the invasive instrument 10. Through this display and/or optical guide arrangement the data received as described above is forwarded to the operating person. The optical guide arrangement 50 comprises one or more light sources, e.g. laser(s), with lenses and/or mirrors, or diffractive optical elements, preferably as an optical guide unit 51 built-in inside the head 105 of the X-ray apparatus, or as a separate optical guide unit 52 that can be connected to the head 105 when needed. The built-in optical guide unit 51 and the X-ray source 101 are preferably provided with such a construction that the X-ray source can be moved in a direction D_{-X}, e.g. a horizontal direction, into a waiting position to make room for movement D_{+O} of the optical guide unit into a guide position where the X-ray source originally was, and that the X-ray source can be moved in an opposite direction D_{+X} to its original position for radiation, whereupon the optical guide unit moves in the opposite direction D_{-O} into its standby position. Typically the directions D_{-O}, D_{+O}, D_{+X} and D_{-X} are parallel. This arrangement, called as a park-back function, is practical for attaining coincidence of the focus points of the X-ray source and the optical guide unit. The optical guide unit 51 or 52 produces at least one light beam 60, which is directed into said tissue surface 3 and within the surface area accessible for insertion of the invasive instrument 10. The light beam, when hitting the tissue surface, preferably has a form of a cross K1, K2, as visible in FIGS. 1B, 2B and 8. It shall be understood that also other, more complicated cross-sectional forms for the light beam or combinations of light beams can be used. Fixing the optical guide unit 51 or 52 to the head 105, especially positioning the optical guide unit 51 within the head, provides substantial advantages, because the focus point of the X-ray beam and the focus point of the light beam(s) can be arranged to be coincident, and the tilt angle α of the light beam 60 and the radiation directions D1, D2, D3 can be matched without problems. The optical guide unit 51 or 52 has means to transfer the position of the light beam and the cross K1, K2 at least in the directions of planar coordinates r, φ, or preferably x, y parallel to the platform. Tilting of the head 105 around the axis 102 that is parallel to the platform produces various values for the tilt angle α, which are preferably the only data of the moving direction P1 of the invasive instrument. To enable using tilt angle only, it is required that the entering point E of the invasive instrument is selected to be in the plane 70 that goes through the lesion T and is perpendicular to the axis 102, and that the invasive instrument is inserted in a direction, which is in this plane 70. This is actually not a limitation, because any point outside this plane 70 is not closer to the lesion than one or two points in this plane. In this case, the cross K1 has lines that are parallel to the x- and y-coordinate axes, and simultaneously parallel and respectively perpendicular to the tilt axis 102. If however, areas outside the plane are wanted to be included for possible entering points a further angle, i.e. the turn angle β, is needed, which means that the light beam producing the cross K2 shall be turnable around its center line, i.e. around e.g. z-coordinate axis. In this case the invasive instrument can be inserted in any direction, but the optical guide arrangement 50 would be much more complicated. There are several known constructions for the optical guide arrangement 50, and any person skilled in the art is able to design different new constructions on the basis of the features described above, and accordingly, optical guide arrangement is not described more in detail.

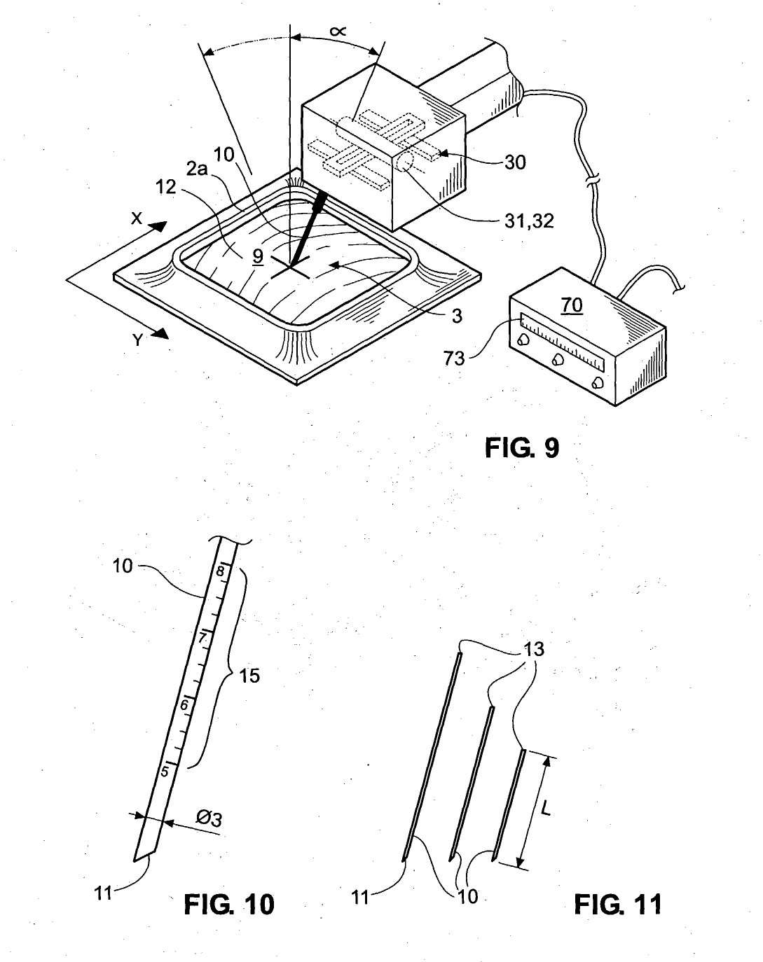

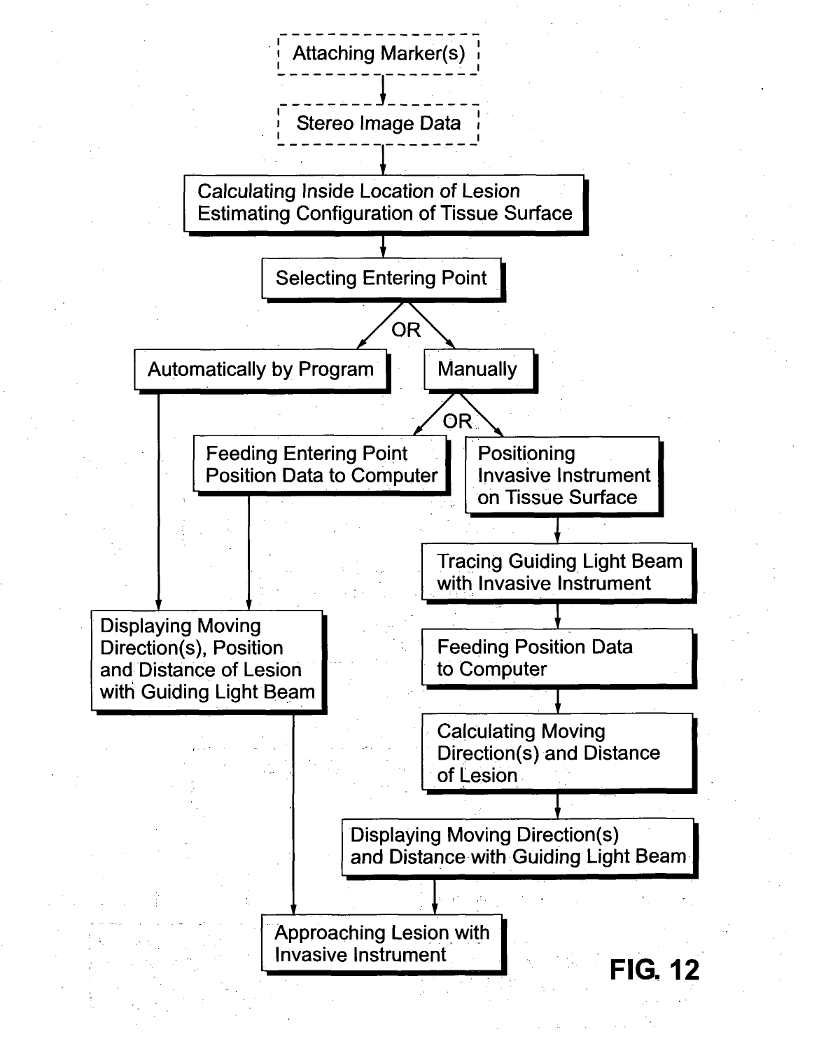

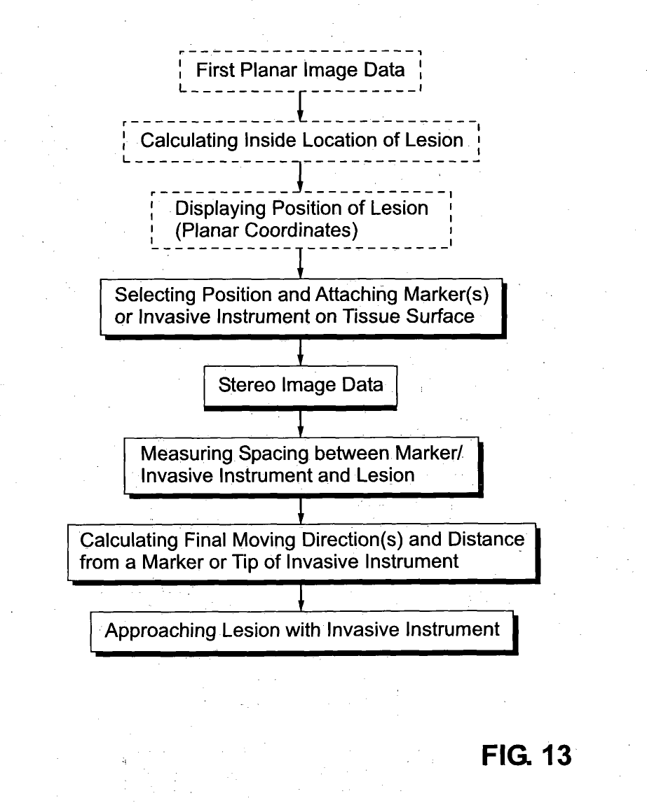

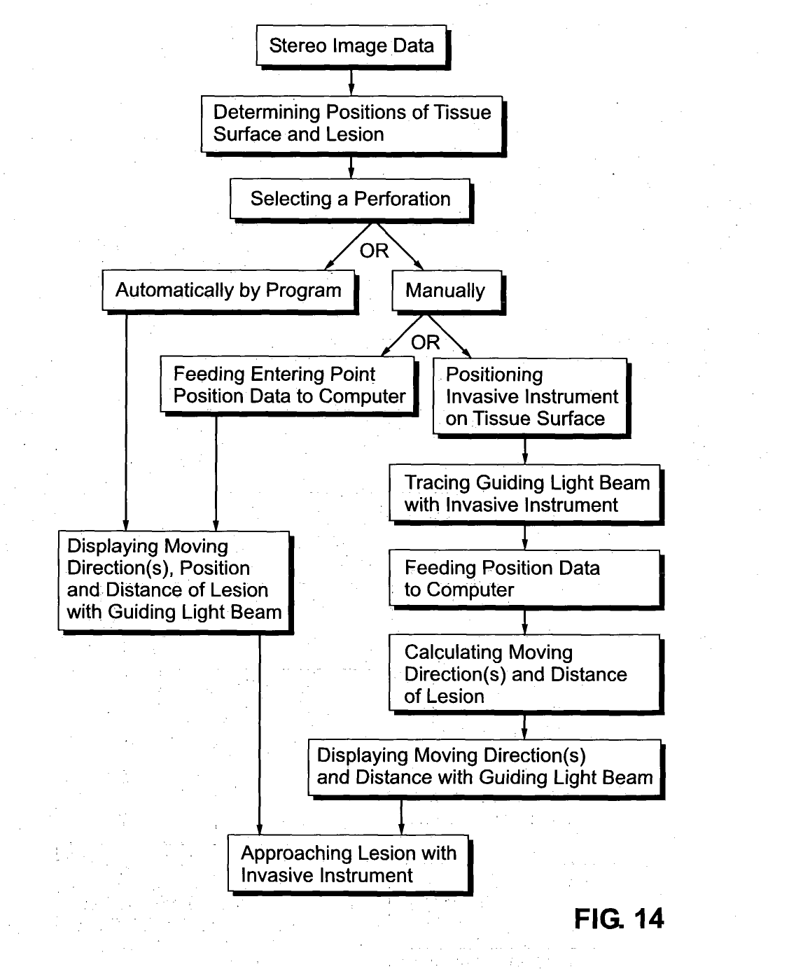

There are several ways how the at least one light beam 60 can be controlled and connected to a control unit or to a combination of a X-ray image analysis computer and a light beam control unit. FIGS. 1A, 1B show the latter alternative, whereupon there is a computer unit 70 comprising both X-ray image analysis computer and the light beam controller connected to optical guide unit 51 or 52. The computer unit 70 is connected to the radiographic image detector 5 to receive the image data and to process the image data as described earlier in this description. The beam control unit of the computer unit can have several types of user interfaces. For the first type of interface the computer comprises a specialized program or algorithm, which searches automatically the potential or possible lesions T from the images=image data, and calculates their positions providing the corresponding three coordinate values x, y, z. Simultaneously the specialized program or algorithm estimates the configuration of the tissue surface 3, whereafter the program/algorithm selects the optimum entering point E and calculates the moving direction, i.e. the tilt angle α, of the invasive instrument and the distance S between the entering point and the lesion. These values are forwarded into the light beam controller in the computer unit, which transfers the light beam 60, via the optical guide unit 51, 52, in the directions of the coordinate axes parallel to the platform to have the calculated coordinate values x, y of the entering point so displaying or indicating the entering point E for the operating person, and tilts the head 105 to have the calculated tilt angle α so displaying or indicating the moving direction P1 of the invasive instrument for the operating person as shown in FIG. 8. For the preferred second type of interface the operating person evaluates image(s) or other data, displayed in any known or new way not shown in the figures, using his/hers personal experience, and decides which are those lesions to be further examined and also selects the entering point E for the invasive instrument. The operating person feeds the position data of both the lesion T and the entering point into the computer unit, e.g. through the keyboard 71. A program/algorithm calculates the moving direction, i.e. the tilt angle α, of the invasive instrument and the distance S between the entering point and the lesion. The same values as above are then forwarded into the light beam controller in the computer unit, which transfers the light beam 60, via the optical guide unit 51, 52, in the directions of the coordinate axes parallel to the platform to have the calculated coordinate values x, y of the entering point so displaying or indicating the entering point E for the operating person, and tilts the head 105 to have the calculated tilt angle α so displaying or indicating the moving direction P1 of the invasive instrument for the operating person as shown in FIG. 8. For the preferred third type of interface the operating person evaluates image(s) or other data, and decides which are those lesions to be further examined and also selects the entering point E for the invasive instrument, as described above. In the next step, the operating person takes the invasive instrument 10 or marker 4 and positions its tip 11 or it on the tissue surface 3 of the body portion 9 at the entering point E selected by herself/himself. Then the operating person feeds the position data of both the lesion T into the computer unit, as described above, and transfers the light beam 60, using e.g. the control buttons 72 in the computer unit, to alignment with the entering point indicated either by the invasive instrument 10 or by the marker 4. This step is called tracing, and in the context thereof the light beam controller simultaneously feeds the coordinate values x, y of the entering point, these are actually introduced by the operation of the control buttons, to the computer unit. Next a program/algorithm calculates the moving direction, i.e. the tilt angle α, of the invasive instrument and the distance S between the entering point and the lesion. Finally the light beam controller in the computer tilts the head 105 to have the calculated tilt angle α so displaying or indicating the moving direction P1 of the invasive instrument for the operating person as shown in FIG. 8. It is also possible that the operating person tilts the head 105 manually after receiving the necessary value of the tilt angle α. The alignment of the invasive instrument 10 with the light beam 60 is readily visible when the light spot 61 caused by the center of the cross K1, K2 is on the outer end 13 of the invasive instrument, as shown in FIG. 8, and the non-alignment is readily visible by the lack of the light spot. The optical guide unit 51, 52 comprises either motors to move the light beam into that position and angle, data for which being provided by the computer unit, or in the second type, light beam position means, which forward that position data of the beam traced to match with the marker or the tip of the invasive instrument into the computer unit, further in the third type, adjustment means for manual tracing. The motors, adjustment means and light beam position detectors utilized can be of any known or new type, and accordingly, they are not described in detail.

The control of the insertion depth, i.e. the control of the calculated distance S can be performed also in several ways. The traditional way is to use an invasive instrument with length indicia forming a scale 15, as shown in FIG. 10. This is a very simple and cheap arrangement, but the visibility of the scale indicia is not good in practice causing inaccuracy in insertion. Another way is to use a set of invasive instruments 10, whereupon each individual instrument has a predetermined length, as shown in FIG. 11. When the distance S is displayed to the operating person, she/he selects an individual invasive instrument having the corresponding length L, which individual instrument is then inserted through the entering point and forwarded to a total depth so that the outer end 13 is in the level of the tissue surface. A great number of invasive instruments are needed in order to have any length L with a difference of e.g. 1 mm therebetween available. A third way is to use one or a few invasive instruments 10 with a predetermined length L or predetermined lengths L with greater length difference of e.g. a couple of centimeters or an inch, and a flabellate light beam 65. The flabellate light beam 65 is flat, having e.g. a thickness not greater than 1 mm, in the direction perpendicular to the platform 1 and a substantial width in the directions parallel to platform. After calculating the distance S for the invasive instrument, the flabellate light beam 65 is transferred by the computer unit 70 to a level H on top of the tissue surface 3 to have the coordinate value z, which equal to projection of the length L of the invasive instrument as tilted in the direction of the platform above the lesion T. This means that level H upwards from the lesion T in direction perpendicular to the platform is H=L×cos α. When the invasive instrument is inserted into the body portion it is readily visible that there is light strip 62 on the invasive instrument, shown in FIG. 8, and that this light strip 62 disappear when the proper distance S is reached, whereupon inserting is stopped.

It is also possible to use instrument guide device 30, which can be an automatic device comprising position motor means 32 or a stand-by device comprising position detection means 31, as shown in FIG. 9. The alternative with the position detection means 31 is preferred. In that case the invasive instrument 10 having a tip 11 is attached to the guide device 30, more specifically to the position detection means 31 in the device. The position detection means comprise detectors to detect the position of the invasive instrument in the two coordinate directions x, y parallel to the platform, the tilt angle α of the invasive instrument and the moving distance S of the invasive instrument in the direction of its length L, and the computer unit 70, to which the detectors are connected. When the operating person moves the tip of the invasive instrument by manual activation to approach said lesion the detectors provides position and direction data and forward those into the computer unit displaying them on a display 73. The position detection means so allows displaying the two prevailing coordinates, a prevailing direction P1, P2 and a prevailing distance S of said invasive instrument. While moving the tip of the invasive instrument manually towards said lesion T, the operating person simultaneously compares these detected and displayed two prevailing coordinates, the detected prevailing direction and the detected prevailing distance with the calculated two coordinate values, the calculated moving direction and the calculated distance, which calculated values are also displayed on the display 73, and operates so as to minimize the difference therebetween. This procedure can be called a computer aided manual insertion of the invasive instrument. It is also closely analogous to the tracing method described above. The position detection means 31 utilized can be of any known or new type, and accordingly, they are not described in detail.

The alternative with the position motor means 32 the invasive instrument 10 having a tip 11 is attached to the guide device 30, more specifically to the position motor means 32 in the device. The position motor means comprise motors to change the position of the invasive instrument in the two coordinate directions x, y parallel to the platform, the tilt angle α of the invasive instrument and the moving distance S of the invasive instrument in the direction of its length L, and the computer unit 70, to which the motors are connected. In this case the output two coordinate values, the moving direction and the distance is conducted to said position motors, whereupon the computer unit allows the position motor means 32 to move said tip of the invasive instrument to approach and reach the lesion T. The position motor means 32 utilized can be of any known or new type, and accordingly, they are not described in detail.

1. A method for pointing a suspected lesion in an X-rayed body portion of a human or animalian body, the method comprising the steps of:
clamping said body portion in a fixed position on a platform provided with a radiographic imaging detector, said body portion having a substantial non-compressed tissue surface area apart from said platform towards an X-ray source, and the suspected lesion having an inside location within said body portion;
radiating said body portion with X-rays coming successively from at least two different directions to form at least two planar images and respective image data of said body portion;
calculating, from said at least two image data and from said at least two directions, said inside location in a predetermined three-dimensional coordinate system having two coordinate values in a plane substantially parallel to said platform;
estimating a configuration of said tissue surface from said image data;
selecting an entering point for an invasive instrument within said surface area;
determining a moving direction for said invasive instrument;
calculating a distance between said entering point on said estimated surface area and said calculated inside location in said moving direction; and
displaying or outputting said moving direction and said distance, and tracing or
displaying or outputting said two coordinate values, for guiding said invasive instrument.

2. A method in accordance with claim 1, wherein said predetermined three-dimensional coordinate system is an orthogonal coordinate system or a polar coordinate system or a combination of an orthogonal and a polar coordinate system.

3. A method in accordance with claim 1, wherein said moving direction includes at least a tilt angle with an axis line parallel to said platform.

4. A method in accordance with claim 3, further comprising the steps of:
determining said moving direction to be in a first plane perpendicular to said platform and parallel to one of said two coordinate values; and
displaying or outputting said tilt angle solely as the moving direction.

5. A method in accordance with claim 3, wherein said moving direction further includes a turn angle with an axis line perpendicular to said platform.

6. A method in accordance with claim 5, further comprising the steps of:
determining said moving direction to be in a second plane perpendicular to said platform and non-parallel to any of said two coordinate values; and
displaying or outputting both said tilt angle and said turn angle as the moving direction.

7. A method in accordance with claim 1, further comprising the steps of:
providing and directing at least one light beam into said tissue surface and within said surface area, said light beam being movable at least parallel to said platform and tiltable around an axis line parallel to said platform;
moving said light beam to alignment with said selected entering point; and
tilting said light beam to alignment with said determined moving direction.

8. A method in accordance with claim 7, further comprising the step of turning said at least one light beam around an axis line perpendicular to said platform to alignment with said determined moving direction.

9. A method in accordance with claim 7, further comprising the steps of:
providing and directing a flattened light beam above said tissue surface and parallel to said platform, said light beam being movable at least in a direction perpendicular to said platform; and
moving said light beam at a level above said entering point being equal to a difference between the lengths of said invasive instrument and said calculated distance.

10. A method in accordance with claim 7, wherein an invasive instrument has length indicia or a predetermined length respective to said distance is used.

11. A method in accordance with claim 1, further comprising, prior to said steps of determining moving direction and calculating distance, the steps of:
providing and directing at least one light beam into said tissue surface and within said surface area, said light beam being movable at least parallel to said platform and tiltable around an axis line parallel to said platform and connected to light beam position means;
selecting manually said entering point;
positioning said invasive instrument having a tip on said tissue surface at said selected entering point;
tracing said light beam to alignment with said tip; and
allowing said light beam position means to output two prevailing coordinates of said entering point for said determining of said moving direction and said calculating of said distance.

12. A method in accordance with claim 1, further comprising, prior to said steps of determining moving direction and calculating distance, the steps of:
providing and directing at least one light beam into said tissue surface and within said surface area, said light beam being movable at least parallel to said platform and tiltable around an axis line parallel to said platform and connected to position detection means;
selecting manually said entering point;
feeding two coordinate values of said entering point being in a plane substantially parallel to said platform for said determining of said moving direction and said calculating of said distance.

13. A method in accordance with claim 1, further comprising the steps of:
providing an invasive instrument guide device with position detection means;
attaching the invasive instrument having a tip to said guide device and in connection with said position detection means;
allowing said position detection means to display two prevailing coordinates, a prevailing direction and a prevailing distance of said invasive instrument; and
moving said tip of the invasive instrument by manual activation to approach said lesion by comparing said displayed two prevailing coordinates, said prevailing direction and said prevailing distance with said calculated two coordinate values, moving direction and distance, and minimizing a difference therebetween.

14. A method in accordance with claim 1, further comprising the steps of:
providing an invasive instrument guide device with position motor means;
attaching the invasive instrument having a tip to said guide device and in connection with said position motor means;
conducting said outputted two coordinate values, said moving direction and said distance to said position motor means; and
allowing said position motor means to move said tip of the invasive instrument to approach said lesion.

15. A method for pointing a suspected lesion in an X-rayed body portion of a human or animalian body, the method comprising the steps:
clamping said body portion in a fixed position on a platform provided with a radiographic imaging detector, said body portion having a substantial non-compressed tissue surface area apart from said platform towards an X-ray source, and the suspected lesion having an inside location within said body portion;
attaching at least one marker on said tissue surface area to have an outside location;
radiating said body portion with X-rays coming successively from at least two different directions to form at least two planar images and respective image data of said body portion;
deriving inside location data and outside location data from said at least two image data and from said at least two directions;
calculating said inside location in a predetermined three-dimensional coordinate system from said inside location data with two coordinate values in a plane substantially parallel to said platform;
estimating a configuration of said tissue surface from said outside location data;
selecting an entering point for an invasive instrument within said surface area;
determining a moving direction for said invasive instrument;
calculating a distance between said estimated tissue surface and said calculated inside location in said moving direction; and
displaying or outputting said moving direction and said distance, and tracing or
displaying or outputting said two coordinate values, for guiding said invasive instrument.

16. A method in accordance with claim 15, wherein said predetermined three-dimensional coordinate system is an orthogonal coordinate system or a polar coordinate system or a combination of an orthogonal and a polar coordinate system.

17. A method in accordance with claim 15, wherein said moving direction includes at least a tilt angle with an axis line parallel to said platform.

18. A method in accordance with claim 17, further comprising the steps of:
determining said moving direction to be in a first plane perpendicular to said platform and parallel to one of said two coordinate values; and
displaying or outputting said tilt angle solely as the moving direction.

19. A method in accordance with claim 18, wherein said moving direction further includes a turn angle with an axis line perpendicular to said platform.

20. A method in accordance with claim 19, further comprising the steps of:
determining said moving direction to be in a second plane perpendicular to said platform and non-parallel to any of said two coordinate values; and
displaying or outputting both said tilt angle and said turn angle as the moving direction.

21. A method in accordance with claim 15, further comprising the steps of:
providing and directing at least one light beam into said tissue surface and within said surface area, said light beam being movable at least parallel to said platform and tiltable around an axis line parallel to said platform;
moving said light beam to alignment with said selected entering point; and
tilting said light beam to alignment with said determined moving direction.

22. A method in accordance with claim 21, further comprising the step of turning said at least one light beam around an axis line perpendicular to said platform to alignment with said determined moving direction.

23. A method in accordance with claim 21, further comprising the steps of:
providing and directing a flattened light beam above said tissue surface and parallel to said platform, said light beam being movable at least in a direction perpendicular to said platform; and
moving said light beam at a level above said entering point being equal to a difference between the length of said invasive instrument and said calculated distance.

24. A method in accordance with claim 21, wherein an invasive instrument has length indicia or a predetermined length respective to said distance is used.

25. A method in accordance with claim 15, further comprising, prior to said steps of determining moving direction and calculating distance, the steps of:
providing and directing at least one light beam into said tissue surface and within said surface area, said light beam being movable at least parallel to said platform and tiltable around an axis line parallel to said platform and connected to light beam position means;
selecting manually said entering point;
positioning said invasive instrument on said tissue surface at said selected entering point;
tracing said light beam to alignment with said invasive instrument; and
allowing said light beam position means to output two prevailing coordinates of said invasive instrument for said determining of said moving direction and said calculating of said distance.

26. A method in accordance with claim 15, further comprising, prior to said steps of determining moving direction and calculating distance, the steps of:
providing and directing at least one light beam into said tissue surface and within said surface area, said light beam being movable at least parallel to said platform and tiltable around an axis line parallel to said platform and connected to light beam position means;
selecting manually said entering point;
feeding two coordinate values of said entering point being in a plane substantially parallel to said platform for said determining of said moving direction and said calculating of said distance.

27. A method in accordance with claim 15, further comprising the steps of:
providing an invasive instrument guide device with position detection means;
attaching the invasive instrument having a tip to said guide device and in connection with said position detection means;
allowing said position detection means to display two prevailing coordinates, a prevailing direction and a prevailing distance of said invasive instrument; and
moving said tip of the invasive instrument by manual activation to approach said lesion by comparing said displayed two prevailing coordinates, said prevailing direction and said prevailing distance with said calculated two coordinate values, moving direction and distance, and minimizing a difference therebetween.

28. A method in accordance with claim 15, further comprising the steps of:
providing an invasive instrument guide device with position motor means;
attaching the invasive instrument having a tip to said guide device and in connection with said position motor means;
conducting said outputted two coordinate values, said moving direction and said distance to said position motor means; and
allowing said position motor means to move said tip of the invasive instrument to approach said lesion.

29. A method for pointing a suspected lesion in an X-rayed body portion of a human or animalian body, the method comprising the steps:
clamping said body portion in a fixed position on a platform provided with a radiographic imaging detector, said body portion having a substantial non-compressed tissue surface area apart from said platform towards an X-ray source, and the suspected lesion having an inside location within said body portion;
attaching at least one marker on said tissue surface to have an outside location;
radiating said body portion with X-rays coming successively from at least two different directions to form at least two individual images and respective image data of said body portion;
deriving inside location data and outside location data from said at least two image data and from said at least two directions;
calculating a direction and a respective distance between said marker and said calculated inside location for entering an invasive instrument; and
displaying or outputting said direction and said distance for guiding said invasive instrument.

30. A method in accordance with claim 29, wherein said predetermined three-dimensional coordinate system is an orthogonal coordinate system or a polar coordinate system or a combination of an orthogonal and a polar coordinate system.

31. A method in accordance with claim 29, wherein said direction is a moving direction for said invasive instrument including at least a tilt angle with an axis line parallel to said platform.

32. A method in accordance with claim 30, further comprising the steps of:
attaching said marker on said tissue surface so as to be in a first plane perpendicular to said platform and parallel to one of said two coordinate values;
determining said moving direction to be in said first plane; and
displaying or outputting said tilt angle solely as the moving direction.

33. A method in accordance with claim 30, wherein said moving direction further includes a turn angle with an axis line perpendicular to said platform.

34. A method in accordance with claim 30, further comprising the steps of:
attaching said marker on said tissue surface so as to be in a second plane perpendicular to said platform and non-parallel to any of said two coordinate values;
determining said moving direction to be in said second plane; and
displaying or outputting both said tilt angle and said turn angle as the moving direction.

35. A method in accordance with claim 29, further comprising the steps of:
providing and directing at least one light beam into said tissue surface and within said surface area, said light beam being movable at least parallel to said platform and tiltable around an axis line parallel to said platform;
moving said light beam to alignment with said marker; and
tilting said light beam to alignment with said calculated direction.

36. A method in accordance with claim 35, further comprising the step of turning said at least one light beam around an axis line perpendicular to said platform to alignment with said determined moving direction.

37. A method in accordance with claim 35, further comprising the steps of:
providing and directing a flattened light beam above said tissue surface and parallel to said platform, said light beam being movable at least in a direction perpendicular to said platform; and
moving said light beam at a level above said entering point being equal to a difference between the lengths of said invasive instrument and said calculated distance.

38. A method in accordance with claim 35, wherein an invasive instrument has length indicia or a predetermined length respective to said distance is used.

39. A method in accordance with claim 29, further comprising the steps of:
providing an invasive instrument guide device with position detection means;
attaching the invasive instrument having a tip to said guide device and in connection with said position detection means;
allowing said position detection means to display a prevailing direction and a prevailing distance of said invasive instrument;
moving said tip of the invasive instrument by manual activation from the side of the marker to approach said lesion by comparing said prevailing direction and said prevailing distance with said calculated direction and distance, and minimizing a difference therebetween.

40. A method for pointing a suspected lesion in an X-rayed body portion of a human or animalian body, the method comprising the steps:
clamping said body portion in a fixed position on a platform provided with a radiographic imaging detector, said body portion having a substantial non-compressed tissue surface area apart from said platform towards an X-ray source, and the suspected lesion having an inside location within said body portion;
radiating said body portion with X-rays coming from at least a first direction to form at least a first individual image and respective image data of said body portion;
deriving inside location data from said at least first images and from said at least first direction;
calculating said inside location in a predetermined two-dimensional coordinate system from said inside location data with coordinate values in a plane substantially parallel to said platform;
displaying or outputting said two coordinates for guiding said invasive instrument;
determining a moving direction for an invasive instrument having a tip;
radiating said body portion, after inserting an invasive instrument into said body portion or in contact or approaching a contact with said tissue surface, with X-rays coming from at least a second direction to form at least a second individual image of said body portion;
measuring a spacing between said tip and said suspected lesion from said second image;
calculating, from said spacing and from said second direction, a distance between said tip and said suspected lesion in said moving direction; and
displaying or outputting said distance and said moving direction for guiding said invasive instrument.

41. A method in accordance with claim 40, wherein said moving direction includes at least a tilt angle with an axis line parallel to said platform.

42. A method in accordance with claim 41, further comprising the steps of:
determining said moving direction to be in a first plane perpendicular to said platform and parallel to one of said two coordinate values; and
displaying or outputting said tilt angle solely as the moving direction.

43. A method in accordance with claim 41, wherein said moving direction further includes a turn angle with an axis line perpendicular to said platform.

44. A method in accordance with claim 43, further comprising the steps of:
determining said moving direction to be in a second plane perpendicular to said platform and non-parallel to any of said two coordinate values; and
displaying or outputting both said tilt angle and said turn angle as the moving direction.

45. A method in accordance with claim 40, further comprising the steps of:
providing and directing at least one light beam into said tissue surface and within said surface area, said light beam being movable at least parallel to said platform and tiltable around an axis line parallel to said platform;
tracing said light beam to alignment with said tip in contact with said tissue surface, or to alignment with a point where said invasive instrument crosses said tissue surface; and
tilting said light beam to alignment with said determined moving direction.

46. A method in accordance with claim 45, further comprising the step of turning said at least one light beam around an axis line perpendicular to said platform to alignment with said determined moving direction.

47. A method in accordance with claim 45, further comprising the steps of:
providing and directing a flattened light beam above said tissue surface and parallel to said platform, said light beam being movable at least in a direction perpendicular to said platform; and
moving said light beam at a level above said entering point being equal to a difference between the lengths of said invasive instrument and said calculated distance.

48. A method in accordance with claim 45, wherein an invasive instrument has length indicia or a predetermined length respective to said distance is used.

49. A method for pointing a suspected lesion in an X-rayed body portion of a human or animalian body, the method comprising the steps:
clamping said body portion in a fixed position on a platform provided with a radiographic imaging detector, said body portion having a substantial tissue surface area apart from said platform and compressed by a compression plate substantially transparent to X-rays and having a plurality of perforations towards an X-ray source, and the suspected lesion having an inside location within said body portion;
radiating said body portion with X-rays coming successively from at least two different directions to form at least two individual images and respective image data of said body portion and of said perforated plate;
deriving inside location data and perforated plate location data from said at least two image data and from said at least two directions;
electing at least one perforation in said plate and determining a moving direction for an invasive instrument through said at least one perforation;
calculating a distance between said at least one perforation of said plate and said calculated inside location in said moving direction of the invasive instrument; and
displaying or outputting said at least one perforation, said direction and said distance for guiding said invasive instrument.

50. A method in accordance with claim 49, wherein said moving direction includes at least a tilt angle with an axis line parallel to said platform.

51. A method in accordance with claim 50, further comprising the steps of:
determining said moving direction to be in a first plane perpendicular to said platform and parallel to one of said two coordinate values; and
displaying or outputting said tilt angle solely as the moving direction.

52. A method in accordance with claim 50, wherein said moving direction further includes a turn angle with an axis line perpendicular to said platform.

53. A method in accordance with claim 52, further comprising the steps of:
determining said moving direction to be in a second plane perpendicular to said platform and non-parallel to any of said two coordinate values; and
displaying or outputting both said tilt angle and said turn angle as the moving direction.

54. A method in accordance with claim 49, further comprising the steps of:
providing and directing at least one light beam into said tissue surface and within said surface area, said light beam being movable at least parallel to said platform and tiltable around an axis line parallel to said platform;
moving said light beam to alignment with said selected perforation; and
tilting said light beam to alignment with said determined moving direction.

55. A method in accordance with claim 54, further comprising the step of turning said at least one light beam around an axis line perpendicular to said platform to alignment with said determined moving direction.

56. A method in accordance with claim 49, further comprising, prior to said steps of determining moving direction and calculating distance, the steps of:
providing and directing at least one light beam into said tissue surface and within said surface area, said light beam being movable at least parallel to said platform and tiltable around an axis line parallel to said platform and connected to light beam position means;
selecting manually said perforation;
positioning said invasive instrument on said tissue surface at said selected perforation;
tracing said light beam to alignment with said invasive instrument; and
allowing said light beam position means to output two prevailing coordinates of said selected perforation for said determining of said moving direction and said calculating of said distance.

57. A method in accordance with claim 49, further comprising, prior to said steps of determining moving direction and calculating distance, the steps of:
providing and directing at least one light beam into said tissue surface and within said surface area, said light beam being movable at least parallel to said platform and tiltable around an axis line parallel to said platform and connected to light beam position means;
selecting manually said perforation;
feeding two coordinate values of said selected perforation being in a plane substantially parallel to said platform for said determining of said moving direction and said calculating of said distance.

58. A method in accordance with claim 54, further comprising the steps of:
providing and directing a flattened light beam above said tissue surface and parallel to said platform, said light beam being movable at least in a direction perpendicular to said platform; and
moving said light beam at a level above said perforation being equal to a difference between the lengths of said invasive instrument and said calculated distance.

59. A method in accordance with claim 54, wherein an invasive instrument has length indicia or a predetermined length respective to said distance is used.

60. A method for pointing a suspected lesion in an X-rayed body portion of a human or animalian body, the method comprising the steps:
clamping said body portion in a fixed position on a platform provided with a radiographic imaging detector, said body portion having a substantial tissue surface area apart from said platform and compressed by a compression plate substantially transparent to X-rays and having a plurality of perforations towards an X-ray source, and the suspected lesion having an inside location within said body portion;
radiating said body portion with X-rays coming from at least a first direction to form at least an individual image and respective image data of said body portion and of said perforated plate;
selecting a perforation in said plate and determining a moving direction for an invasive instrument having a tip through said at least one perforation;
radiating said body portion, after inserting said invasive instrument, with X-rays coming from at least a second direction to form at least another individual image and respective image data of said body portion and of said perforated plate and said invasive instrument;
calculating a distance between said tip of the invasive instrument and said calculated inside location in said moving direction of the invasive instrument;
